(19) United States
(12) Patent Application Publication
(10) Pub. No.: US 2022/0203340 A1
(43) Pub. Date: Jun. 30, 2022

(54) LIGHT PARAFFIN DEHYDROGENATION CATALYSTS AND THEIR APPLICATION IN FLUIDIZED BED DEHYDROGENATION PROCESSES

(71) Applicant: UOP LLC, Des Plaines, IL (US)

(72) Inventors: Avram M. Buchbinder, Chicago, IL (US); Wei Pan, Hoffman Estates, IL (US); J. W. Adriaan Sachtler, Des Plaines, IL (US); Jeffrey M. Noga, Woodridge, IL (US); Xi Zhao, Arlington Heights, IL (US); Ling Zhou, Palatine, IL (US); John A. Karch, Lake Zurich, IL (US)

(21) Appl. No.: 17/137,576

(22) Filed: Dec. 30, 2020

(51) Int. Cl. B01J 23/62; B01J 23/58; B01J 21/04; B01J 21/12; B01J 21/06; B01J 35/02; B01J 35/10; B01J 35/00; B01J 23/96; B01J 38/30; B01J 8/18; B01J 8/26; C07C 5/48

(52) U.S. Cl. CPC B01J 23/626; C07C 2521/06; B01J 21/04; B01J 21/12; B01J 21/063; B01J 35/026; B01J 35/1014; B01J 35/1019; B01J 35/0053; B01J 23/96; B01J 38/30; B01J 8/1827; B01J 8/26; C07C 5/48; C07C 2523/58; C07C 2523/62; C07C 2521/04; C07C 2521/12; B01J 23/58

(57) ABSTRACT

A process is provided for dehydrogenating a paraffinic hydrocarbon comprising sending the paraffinic hydrocarbon to a fluidized bed reactor to be contacted at dehydrogenation reaction conditions with a catalyst composition comprising less than about 0.0999 wt % platinum and about 0.05-2.5 wt % Group I or Group II elements or a mixture thereof. The catalytic composition is prepared without addition of tin, gallium, indium, germanium or lead.

### FIELD OF THE INVENTION

This invention relates generally to a new catalytic material and a process for the dehydrogenation of hydrocarbons using the catalytic material.

### BACKGROUND OF THE INVENTION

Petroleum refining and petrochemical processes frequently involve the selective conversion of hydrocarbons with a catalyst. For example, the dehydrogenation of hydrocarbons is an important commercial process because of the great demand for dehydrogenated hydrocarbons for the manufacture of various chemical products such as detergents, high octane gasolines, pharmaceutical products, plastics, synthetic rubbers, and other products well known to those skilled in the art. One example of this process is dehydrogenating isobutane to produce isobutylene which can be polymerized to provide tackifying agents for adhesives, viscosity-index additives for motor oils, impact-resistant and antioxidant additives for plastics and a component for oligomerized gasoline. Another example of this process is dehydrogenating propane to produce propylene which can be polymerized to produce polypropylene or used for other applications.

The prior art is cognizant of various catalytic composites which contain a Group VIII noble metal component, an alkali or alkaline earth metal component, and a component selected from the group consisting of tin, germanium, lead, indium, gallium, thallium, or mixtures thereof. U.S. Pat. Pub. No. 2005/0033101 and U.S. Pat. No. 6,756,340, both assigned to the present applicant and the entirety of both which are incorporated herein by reference, describe various catalysts that are useful, efficient, and effective for the selective conversion of hydrocarbons.

Fluidized bed processes for dehydrogenation of alkanes have advantages such as enabling more isothermal catalyst bed profiles and higher conversion and minimizing losses to thermal cracking. Fluidized bed processes have shorter catalyst residence time than fixed and moving bed processes, thus faster catalyst deactivation can be tolerated. Given the loosening of catalyst deactivation constraints, catalyst compositions which are less costly may be more feasible.

Thus, there remains an ongoing and continuous need for new catalytic material for selective hydrocarbon conversion processes, especially those that improve on one or more characteristics of the known catalytic compositions, and/or enable new energy efficient processes such as dehydrogenation in a fluidized bed.

### SUMMARY OF THE INVENTION

The present invention provides a new catalytic material, a process for the selective conversion of hydrocarbon using the new catalytic material, as well as a process for regenerating the new catalytic material.

Therefore, the present invention may be characterized, in at least one aspect, as providing a catalyst for a selective conversion of hydrocarbons such as alkanes comprising: a first component consisting of a low amount of platinum with levels below 0.0999 wt % on a volatile-free basis, preferably less than 0.0600 wt % and more preferably less than 0.0400 wt %. It has been found that higher amounts of platinum are not advantageous in performance with a cost savings provided by the low levels that are used. A second component is from 0.05 to 2.5 wt % of one or more of Group I or Group II elements. Preferably the second component is present at amounts of 0.1 to 0.4 wt %. The ratio of the second component to the first component is higher than in the prior art because the catalyst does not contain tin or other modifiers. Specifically, the platinum and Group I and Group II elements are present at an atomic ratio of about 1:20 to 1:200. The catalyst composition is low in chlorides and comprises less than about 1000 ppm by weight chloride. The catalyst is made without addition of tin, gallium, indium, germanium, lead or chromium.

The catalyst may further comprise an alumina support for the forming of catalyst particles having a total pore volume of from 0.2 cm³/g to about 0.8 cm³/g, particle size of from 20 micrometers to 200 micrometers with median particle size of from 50 micrometers to 150 micrometers. The catalyst has a surface area of about 60 to about 250 m²/g and a bulk density of about 0.7 to about 1.1 g/cm³.

In at least one other aspect, the present invention may be characterized as providing a process for regenerating a catalyst used for a selective conversion of hydrocarbons comprising: removing coke from a catalytic composite having a first component selected from the group consisting of platinum, a second component selected from the group consisting of Group I and Group II elements.

In another aspect, the present invention may be characterized as providing a process for the selective conversion of hydrocarbons comprising contacting a hydrocarbon at selective conversion conditions with the catalyst composition of this invention. Additional aspects, embodiments, and details of the invention, all of which may be combinable in any manner, are set forth in the following detailed description of the invention.

The catalyst is made without the addition of tin, gallium, indium, germanium or lead and contains only inadvertently added amounts of less than about 500 ppm by weight of these elements, preferably less than 100 ppm by weight. Furthermore, the catalyst is made without addition of chromium, and in any event such element is present in amounts less than about 100 ppm by weight.

### DETAILED DESCRIPTION OF THE INVENTION

As described above, a new catalytic material, a process for the selective conversion of hydrocarbon using the new catalytic material, as well as a process for regenerating the new catalytic material have been invented.

In particular, in some processes for alkane dehydrogenation, a fluidized bed propane dehydrogenation design has been found to be advantageous. In a common embodiment, the alkane feed for dehydrogenation is either propane or butane. In connection with such processes for propane dehydrogenation, it has become desirable to increase propylene selectivity which can be reduced due to thermal cracking. Thermal cracking can be minimized in a circulating fluidized bed process, where the heat of reaction is supplied by hot catalyst rather than by heaters. In addition, such processes can address capacity limitations found in the reactor sizes that are used. It has been found important to have a more active and selective catalyst that is the subject of the present invention. When compared to the prior art, these catalysts are regenerable and providing stable multi-cycle light paraffin dehydrogenation performance in fluidized dehydrogenation processes. These catalysts are comprised of a platinum level<0.0999 wt % on a volatile-free basis, preferably less than 0.0600 wt %, and more preferably less than 0.0400 wt % on a volatile-free basis. Low platinum levels are advantageous because of savings in cost and since it has been found that higher platinum levels do not provide any added benefit on aged catalyst since the additional platinum is not active. However, some amount of platinum is needed to maintain desired activity. These catalysts comprise at least 0.0050 wt % Pt, preferably at least 0.0100% Pt and more preferably at least 0.0200% Pt. The catalyst is comprised of 0.05-2.5 wt % one of Group I or Group II elements, preferably 0.1-0.4 wt %. The Group I or Group II element/Pt mole ratio is higher than prior art because the catalyst does not contain tin or other modifiers. The preferable range of platinum to Group I or Group II atomic mole ratio is 1:20-1:200. More preferably the range of platinum to Group I or Group II atomic mole ratio is 1:40-1:90. If the element is lithium, the catalyst preferably contains 0.5-2.5 wt % lithium and the support is a lithium aluminate.

These catalysts do not contain some elements found in prior art catalysts and in particular do not include tin, gallium, indium, germanium, or lead. In the event that there are trace amounts of these elements, the level of these elements in these catalysts should be less than 500 ppm by weight, preferably less than 100 ppm, and further preferably less than 1 ppm in the event that chemicals, supports, and process equipment used to make or handle these catalysts introduce these elements as impurities. Surprisingly, addition of these elements can interfere with regeneration of the catalysts of the present invention in this process. In addition, the catalysts do not contain halogen elements except for possible impurity levels of less than 1000 ppm by weight and preferably less than 500 ppm by weight if chemicals, supports, and process equipment used to make these catalysts do not introduce such trace amounts of halogens. The presence of halogens causes the catalyst to be less selective. The catalysts of the present invention are able to be regenerated in a regenerator with streams comprising oxygen and steam, and/or carbon dioxide. The catalysts are able to be sent to the fluidized-bed dehydrogenation reactor, generally with an intervening stripping step with an inert gas such as N₂. The process does not have a separate reduction step after regeneration, unlike in some other processes where reduction is needed before a regenerated catalyst is sent back to paraffin dehydrogenation reactor. This feature is also different from a fluidized-bed dehydrogenation process that uses Pt-Ga catalysts where the regenerated catalyst needs to be treated again in dry air or oxygen for extended periods of time as put forth in U.S. Pat. No. 9,834,496B2, U.S. Pat. No. 10,065,905B2, and U.S. Pat. No. 10,277,271B2. In the present invention, the catalyst can be regenerated with about 2.5% oxygen by volume. As noted above, the catalyst does not contain gallium or indium, which are expensive additives, nor do they contain halogens or require halogen-assisted platinum dispersion unlike some prior art processes.

These catalysts provide better propylene selectivity and are compatible with being regenerated in a steam-containing environment and sent to a dehydrogenation reactor without reduction. The catalysts are typically in a shape of micro-sphere with median particle size, defined as diameter, in the range of 20-200 microns and can be used in fluidized bed reactors and regenerators with sufficient mechanical strength. Preferably the median particle size is in the range of 50-150 microns. Preferably the particle size distribution has 10^{th} percentile greater than 20 microns and 90^{th} percentile lower than 200 microns. Catalysts are prepared on supports, preferably comprising alumina, with a surface area between 60-200 m²/g. Preferably, the surface area is between 85-140 m²/g. Most preferably the surface area is between 100-140 m²/g. High surface area allows for higher activity and Pt dispersion. The preferable Pt per surface area is less than 0.04 micromoles of Pt per m² of catalyst surface area (measured by BET method). More preferably the platinum per m² of catalyst surface area is less than 0.02 micromoles of Pt per m² of catalyst surface area. The catalysts have a catalyst bulk density (ABD) preferably in the range of 0.7-1.1 g/cm³

In order to measure the ABD, the substance is put into a receiver of known volume and weight. The catalyst is leveled to the top of the vessel and weighed. ABD is calculated by dividing the mass of the catalyst by the volume of the vessel.

The Group I or Group II component of the present invention may be selected from the group consisting of cesium, rubidium, potassium, sodium, and lithium or from the group consisting of barium, strontium, calcium, and magnesium or mixtures of metals from either or both of these groups. Group I or Group II elements from period four (potassium and calcium) are preferred Group I or Group II components and calcium is the most preferred Group I or Group II component. Typically, the Group I or Group II component is present at a level less than 150 micromoles per gram of catalyst. Preferably the Group I or Group II component is present at a level between 25 and 130 micromoles per gram of catalyst.

The Group I or Group II component may be present as a compound such as the oxide, for example, or combined with the carrier materiel or the support material or with the other catalytic components.

The Group I or Group II component may be incorporated in the catalytic composite in any suitable manner such as, for example, by coprecipitation or co-gelation, by ion exchange or impregnation, admixing with precursors to the catalyst support matrix, or by like procedures either before, while, or after other catalytic components are incorporated. A preferred method of incorporating the Group I or Group II component is to impregnate onto the carrier material with a solution of a soluble potassium or calcium salt. Preferred potassium or calcium salts include potassium nitrate, potassium carbonate, potassium acetate, potassium chloride, potassium hydroxide, calcium nitrate, calcium chloride, or calcium acetate. An additional preferred method is admixing a soluble potassium or calcium salt with precursors to the catalyst support matrix. For instance, an alumina powder and aluminum salt along with water are combined with a potassium or a calcium salt and vigorously mixed to produce a slurry. The slurry is spray dried in a spray drier.

The carrier material or support of the present invention is alumina having the characteristics discussed above. The alumina carrier material may be prepared in any suitable manner from synthetic or naturally occurring raw materials. The carrier may be formed in any desired shape such as spheres, pills, cakes, extrudates, powders, granules, etc, and may be used in any particle size suitable for fluidization. A preferred shape of alumina is the sphere. Additionally, the particle size distribution of the carrier material can be mono-modal, bi-modal, or a mixture thereof. The alumina preferably consists primarily of gamma alumina. The alumina may also contain delta and theta alumina or other phases of alumina.

It is preferred that the alumina component is essentially gamma-alumina. By “essentially gamma-alumina”, it is meant that the powder X-ray diffraction pattern contains primarily the diffuse scattering peaks characteristic of the gamma-alumina phase but importantly may also include X-ray diffraction patterns that are characteristic of the delta-alumina transition phase or mixtures thereof. Similar characteristic X-ray diffraction patterns are shown in the 873K-1173K examples of FIG. 3 in the work by Zhou and Snyder “Structures and Transformation mechanisms of the [eta], [gamma] and [theta] transition aluminas” Acta Cryst. (1991). B47, 617-630. These broad diffuse scattering features of the gamma- and delta-alumina transition phases show considerable similarity and overlap in the observable X-ray diffraction patterns. Here, we assume that as the catalyst is exposed to steam and high temperatures in the regenerator, the essentially gamma-alumina phase will slowly convert over a period of months or years to form the higher thermodynamic stability polymorphs of delta- and/or theta-alumina respectively before eventually converting to alpha-alumina. Thus, the starting amount of delta, theta or alpha alumina and/or the gradual transition towards those phases should be minimized. Due to significant structural disorder, there is presently no objective criteria for assigning and interpreting the early stages of the gamma-alumina to delta-alumina transformations and, from a performance perspective, both are acceptable.

As the transition continues to progress further towards the upper end of the delta-theta-alumina series, a diffraction peak at d=1.80 Å can be observed and measured by means of integrated area which is either very weak or absent in the essentially gamma-alumina phase and increases in intensity as the materials progresses towards theta-phase where it eventually resolves to two more defined diffraction peaks at d=1.80 Å and 1.78 Å. This progression is also associated with a decline in catalyst performance.

To monitor this transition, a value is determined from the experimental X-ray diffraction patterns referred to herein as the “theta-index” value, in order to objectively define the amount of transition from gamma to delta and theta aluminas. To determine this value, the integrated area of the delta/theta peak at d=1.80 Å is measured and compared to the (012) reflection (d=3.48 Å) of NIST certified 676a alpha-alumina intensity standard run under the same scan conditions. The theta index is the integrated area of the d=1.80 Å in the sample in question divided by that in the alpha-alumina standard. The theta index value, associated with the peak intensity attributed to the delta/theta phase in the catalysts of this invention is typically less than 0.045, more typically less than 0.040, preferably less than 0.025 and most preferably less than 0.015. Similarly, conversion to alpha-alumina phase can be tracked in a similar manner and as it is the same structural phase as the NIST certified reference material, the same diffraction peak can be observed and measured for integrated area at d=3.49 Å. Herein called the alpha-index, the preset example, the alpha-alumina is typically less than 0.02, preferably less than 0.01, and most preferable significantly less than 0.01 or not measurable.

Theta indices and alpha indices presented in the examples herein were extracted from diffraction patters that were obtained using standard x-ray powder diffraction techniques where the radiation source was a high-intensity, x-ray tube operated at 40 kV and 40 mA. The diffraction pattern from the copper K-alpha radiation was obtained by appropriate computer-based techniques. Powder samples were pressed flat into a plate and continuously scanned between 5 degrees and 90 degrees (2.theta) with scan time sufficiently long as to minimize background noise in the scan. Interplanar spacings (d) in Angstrom units were obtained from the position of the diffraction peaks expressed as theta, where theta is the Bragg angle as observed from digitized data. Intensities were determined from the integrated area of the diffraction peak after subtracting background. In the case of the peak intensity used to determine the theta-index value, background subtraction can be challenging due to the broad peaks of the gamma- and delta-phases. Here, a linear background is used starting with a suitable minimum around d=1.84 Å and finishing around d=1.76 Å. In cases such as gamma-alumina where intensity in the specified range is at or below the linear-background, the theta-index is taken to be 0. As will be understood by those skilled in the art the determination of the parameter 2.theta is subject to both human and mechanical error, which in combination can impose an uncertainty of about +/−0.4 degree. on each reported value of 2.theta. This uncertainty is also translated to the reported values of the d-spacings, which are calculated from the 2.theta values. Alpha-alumina (corundum structure: Al₂O₃) powder was scanned in the same manner as the samples of interest to provide an intensity reference point. Only a high purity and suitably prepared alumina source must be used. One such choice is the NIST certified Standard Reference Material 676a. Of particular importance is both purity and particle morphology as alumina grains should be sub-micrometer in size and equi-axial in shape to prevent preferred orientation effects when preparing a sample.

In an embodiment, alumina particles are produced by a spray drying process although any process for preparing microparticles of the desired size range is suitable. In a typical spray drying process, an alumina slurry with specific particle size distribution and pH is combined with a binder, well mixed, and pumped into a spray dryer at a controlled rate through either a nozzle or wheel which will atomize the slurry into small liquid droplets. The slurry droplets in contact with hot air are dried to the solid particle product with specific moisture content, particle size distribution, bulk density, and attrition resistance. The said alumina slurry comprises, but is not limited to, boehmite, gamma alumina, aluminum chloride, aluminum chlorohydrate, aluminum phosphate, aluminum sulphate, alkali and alkaline earth metal aluminates. The said binder includes, but not limited to, acid-peptized alumina, aluminum chlorohydrate, colloidal alumina, and silica aluminate.

It may be desirable to add additional additives to the catalyst to promote long-term catalyst stability. Such additives may include but are not limited to Mg, Ca, Sr, Ba, Ti, P, B, and Si. However, some of these additives are noted to result in drastic decreases to catalyst activity or selectivity, so the tradeoff between stability and performance must be considered in selecting a catalyst additive.

As explained, the gamma-alumina form of crystalline alumina is produced from the boehmite or amorphous alumina precursor by closely controlling the maximum calcination temperature experienced by the catalyst support. Calcination temperatures above 500° C. are known to produce alumina comprising essentially crystallites of gamma-alumina. Calcination temperatures of 1100° C. and above are known to promote the formation of alpha-alumina crystallites while temperatures of from 950° to 1100° C. promote the formation of theta-alumina crystallites.

Any suitable method of known in the art can be used to add the catalytic components. The catalytic components are typically added by impregnation to the calcined alumina support. For instance, soluble Pt salts and optionally soluble Group I and or Group II components are dissolved in water. Soluble Pt salts include but are not limited to chloroplatinic acid, tetraamine platinum nitrate, tetraamine platinum chloride and the like. Soluble Group I and Group II components include but are not limited to potassium nitrate, potassium carbonate, potassium acetate, potassium chloride, potassium hydroxide, calcium nitrate, calcium chloride, or calcium acetate. The solution containing the catalytic components is contacted with the support. The contacting can be done by any suitable method known in the art, including wet impregnation, incipient wet impregnation, wet impregnation and evaporation, ion exchange, and the like. In a typical incipient wetness impregnation or pore filling process, the amount of metal solution giving equivalent volume to the total pore volume of the catalyst support, is sprayed on the powder support as it rotates in a rolling equipment, resulting in a free-flowing powder product. The said rolling equipment includes, but not limited to cylinder drum, conical, double cone blender, mixer, and tumbler.

After the catalyst components have been combined with the desired alumina support, the resulting catalyst composite will generally be dried at a temperature of from about 100° to about 320° C. for a period of typically about 1 to 24 hours or more and thereafter calcined at a temperature of about 320° to about 600° C. for a period of about 0.5 to about 10 or more hours. This final calcination typically does not affect the alumina crystallites or ABD. However, the high temperature calcination of the support may be accomplished at this point if desired.

In previous catalysts known in the art, chlorine is added to prevent sintering of catalyst metal components. Surprisingly, addition of chlorine is not needed for this process as catalysts maintain good performance for many cycles with no substantial chlorine on the fresh catalyst and no chlorine added later. In fact, if chlorine is added the selectivity for dehydrogenation is decreased. The catalyst composition is low in chlorides and comprises less than about 1000 ppm by weight chloride, preferably less than 700 ppm chloride and more preferably less than 500 ppm chloride. Similarly, the catalyst also generally does not contain other halogens. Steaming, calcination or washing of the catalyst may be done during catalyst synthesis to remove chlorides that are added during synthesis. These treatments which remove chloride can be done at any stage after a chlorine containing component is added during the synthesis of the catalyst.

According to one or more embodiments, the catalyst composition is used in a hydrocarbon conversion process, such as dehydrogenation. In the preferred process, dehydrogenatable hydrocarbons are contacted with the catalytic composition of the present invention in a dehydrogenation zone maintained at dehydrogenation conditions. This contacting occurs in a fluidized bed system. A fluidized bed system is preferred in one preferred embodiment. The dehydrogenation zone may itself comprise one or more separate reaction zones. The heat required for the endothermic dehydrogenation reaction is primarily provided by the sensible heat of the catalyst that is transferred from the regeneration zone to the reaction zone, although a portion of the heat for the dehydrogenation reaction can come from pre-heating the hydrocarbon feed or preheating a diluent gas.

The hydrocarbon to be converted is preferably an alkane. The alkane is preferably a light alkane such as propane or butane. In an exemplary embodiment the alkane is propane. Hydrocarbons which may be dehydrogenated include dehydrogenatable hydrocarbons having from 2 to 30 or more carbon atoms including paraffins, alkylaromatics, naphthenes, and olefins. One group of hydrocarbons which can be dehydrogenated with the catalyst is the group of paraffins having from 2 to 30 or more carbon atoms. The catalyst is particularly useful for dehydrogenating paraffins having from 3 to 18 or more carbon atoms to the corresponding mono-olefins. The catalyst is especially useful in the dehydrogenation of C2-C6 paraffins, primarily propane and butanes, to mono-olefins.

Dehydrogenation conditions include a temperature of from about 400° to about 900° C., and preferably from 550 to 680° C., more preferably 600 to 640° C., a pressure of from about 0.01 to 10 atmospheres absolute, preferably 0.1 to 3 atmospheres absolute, more preferably 0.75 to 1.5 atmospheres absolute, and a weight hourly space velocity (WHSV) of from about 0.1 to 100 hr⁻¹, preferably 0.5 to 5 hr⁻¹. Generally, for normal paraffins, the lower the molecular weight, the higher the temperature required for comparable conversion. The pressure in the dehydrogenation zone is maintained as low as practicable, consistent with equipment limitations, to maximize the chemical equilibrium advantages.

In fluidized bed processes such as this invention, catalyst is circulated continuously from reactor to regenerator and back to reactor. Catalyst is fluidized in both reactor and regenerator with fluidization gas, which may comprise the alkane reactant, the alkene product, hydrogen, nitrogen or other fluidization gases in the reactor. In the regenerator fluidization gas may comprise air, oxygen, nitrogen, a fuel or other fluidization gases. Generally, the residence time of catalyst particles in the reactor and regenerator is non-uniform and can be described by a distribution of residence times. For definition purposes herein, the residence time distributions of catalyst particles in the reactor are defined on a catalyst weight basis. The average residence time of particles is defined as the mean time spent in the reactor of weight-distribution of catalyst particles. The distribution of catalyst particles in the reactor can have different characteristics. Several fluidized bed process designs known in the art are suitable, including risers, fast fluidized beds, bubbling beds, transport beds, counter-current falling beds, and the like. Different fluidized bed processes have different distributions of catalyst residence times, ranging from plug flow to continuous back-mixed reactors with similar residence time distribution to a continuous stirred tank reactor (CSTR). A preferred embodiment is a fast-fluidized bed with residence time distribution similar to a continuous back-mixed reactor. Since catalyst deactivates quickly under reaction conditions, shorter catalyst residence times allow for higher average catalyst activity since more of the catalyst is at earlier times on stream and is thus more active. This short residence time is critical for enabling the catalysts in this invention. The catalyst in this invention deactivates quickly, but sufficient activity is captured if residence time is short. However, shorter residence times also necessitate faster catalyst circulation rates which over time will lead to more catalyst attrition and require utility costs for circulating catalyst. Preferably, the average catalyst residence time in the reactor is from 30 seconds to 5 minutes. More preferably the average catalyst residence time in the reactor is from 1 minute to 2.5 minutes.

Catalyst deactivation within a catalyst cycle is slower when more catalyst is present in the reactor per unit of feed hydrocarbon. The ratio of catalyst mass flow through the reactor to hydrocarbon feed mass flow through the reactor in a set unit time is often referred to as catalyst to oil ratio. The preferred catalyst to oil ratio is in the range of 10 to 50. The more preferred catalyst to oil ratio is in the range of 15 to 30. The most preferred catalyst to oil ratio is in the range of 20 to 25.

The effluent stream from the dehydrogenation zone generally will contain unconverted dehydrogenatable hydrocarbons, hydrogen, and the products of dehydrogenation reactions. This effluent stream is typically cooled and passed to a hydrogen separation zone to separate a hydrogen-rich vapor phase from a hydrocarbon-rich liquid phase. Generally, the hydrocarbon-rich liquid phase is further separated by means of either a suitable selective adsorbent, a selective solvent, a selective reaction or reactions, or by means of a suitable fractionation scheme. Unconverted dehydrogenatable hydrocarbons are recovered and may be recycled to the dehydrogenation zone. Products of the dehydrogenation reactions are recovered as final products or as intermediate products in the preparation of other compounds or for use as fuel.

The dehydrogenatable hydrocarbons may optionally be admixed with a diluent material before, while, or after being passed to the dehydrogenation zone. The diluent material may be hydrogen, methane, ethane, carbon dioxide, nitrogen, argon, and the like or a mixture thereof. Hydrogen is a preferred diluent. Ordinarily, when hydrogen is utilized as the diluent, it is utilized in amounts sufficient to ensure a diluent-to-hydrocarbon mole ratio of about 0.01:1 to about 40:1, with best results being obtained when the mole ratio range is about 0.01:1 to about 0.5:1. The diluent stream passed to the dehydrogenation zone will typically be recycled diluent separated from the effluent from the dehydrogenation zone in a separation zone. Note that hydrogen is also produced in the reaction. The product hydrogen is not included in the above diluent to hydrocarbon mole ratios.

A small amount of water vapor will be present in the reactor. The water can be present from several sources including but not limited to: being present as a contaminant in the feed, being produced by reacting contaminants in the feed such as reacting methanol to make water, or being carried from the regenerator in gas entrained in the regenerated catalyst stream or adsorbed on the catalyst, or being produced by reacting components of gases entrained in the regenerated catalyst stream such as reacting O₂ to form water. The amount of water in the reactor is preferably less than 2 mol % of the combined products, more preferably less than 0.5 mol %.

To be commercially successful, a dehydrogenation catalyst should exhibit four characteristics, namely: high activity, high selectivity, regenerability, and long term stability. Activity is a measure of the catalyst's ability to convert reactants into products at a specific set of reaction conditions, that is, at a specified temperature, pressure, contact time, and concentration of diluent such as hydrogen, if any. For dehydrogenation catalyst activity, the conversion or disappearance of paraffins in percent relative to the amount of paraffins in the feedstock is measured. In the examples herein, percent conversion of propane is determined by dividing the moles of propane in the product by the moles of propane in the feed, subtracting that number from 1, then multiplying by 100. Selectivity is a measure of the catalyst's ability to convert reactants into the desired product or products relative to the amount of reactants converted. For catalyst selectivity, the amount of olefins in the product, in mole percent of carbon atoms in the product, relative to the total moles of carbon atoms in the paraffins converted is measured. Regenerability is the ability of the catalyst to regain its activity after each regeneration-reaction cycle. To be commercially successful, activity at early time-on-stream in a reaction cycle is similar to activity at early time-on-stream in previous cycles. Later during the reaction cycle the activity will drop due to deactivation, but the activity is restored by the subsequent regeneration cycle. Long term stability is a measure of how stable the catalyst activity and selectivity are over multiple cycles. To be commercially viable, activity after thousands of cycles must be high enough to maintain the desired conversion. Thus, although some loss of activity may be tolerable, catalysts that maintain activity through many cycles are preferred.

In regeneration, carbon deposited on the catalyst as coke during use of the catalyst in a hydrocarbon conversion process is burned off and the catalyst and the catalyst is reactivated to provide a regenerated catalyst with performance characteristics much like the fresh catalyst. In the regenerator, an 02 containing gas such as air is added, and coke and fuel are burned, such that the temperature of the catalyst in the range of 600-800° C., preferably 680-800° C., more preferably 690-750° C. and most preferably 680-730° C. While the amount of oxygen and fuel near the points of injection of oxygen and fuel into the regenerator may be higher, the atmosphere in the regenerator burn zone generally contains 0.5-20 mol % 02, 10-30 mol % steam and 2-8 mol % CO₂. Preferably, the regenerator burn zone contains 0.5-5 mol % O₂. More preferably, the regenerator burn zone contains 1.5-3 mol % O₂. Preferably, the regenerator burn zone contains 15-25 mol % H₂O. Preferably the regenerator burn zone contains less than 0.2% carbon monoxide. Preferably there is little or no remaining fuel in the exit point for gasses from the regenerator. The average residence time of catalyst in the regenerator is preferably less than 2 minutes in order to allow for a small regenerator vessel. It is important that a catalyst for this process have sufficient activity after being subjected to this regeneration condition. The heat source in the regenerator includes burning of coke and burning of a fuel. Typically, the hot catalyst is contacted with nitrogen or inert gas to partially remove O₂, H₂O and CO₂ and returned hot to the reactor. Preferably, the contacting with nitrogen reduces the concentration of O₂, H₂O and CO₂ in the interstitial gas of the regenerated catalyst stream by at least about 80%, more preferably by at least about 90%. No additional reduction step is used, no extra air, dry-air or dry 02 containing gas treatment is needed, and no Pt-redispersion facilitated by Cl is required. The hot catalyst provides most of the heat needed for propane dehydrogenation, and thus the hot catalyst returning to the reactor must have a temperature above the average temperature of catalyst in the reactor. The temperature of the catalyst returning to the reactor is in the range of 600-800° C., preferably in the range of 680-800° C. and most preferably in the range of 680-730° C. One recently developed process for regenerating catalyst may be used in which higher temperature regenerated catalyst is mixed with the lower temperature spent catalyst to heat the spent catalyst together with air or other oxygen to facilitate mixing in the regenerator. The mixing of hot regenerated catalyst with cooler spent catalyst increases the catalyst density in the regenerator and provides sufficient catalyst to absorb heat without excess temperature rise thereby protecting catalyst and equipment. The temperature of the spent catalyst is also increased making the coke on catalyst and the supplemental fuel gas instantly ready to combust without the delay necessary to heat up the spent catalyst to combustion temperature. The regenerated catalyst may be mixed with the spent catalyst before the mixture of catalyst is contacted with the supplemental fuel gas.

The following examples are introduced to further describe the catalyst and process of the invention. These examples are intended as an illustrative embodiment and should not be considered to restrict the otherwise broad interpretation of the invention as set forth in the claims appended hereto.

### EXAMPLES

### Example 1

6.55g of H₂O, 2.46g of 0.15 wt % Pt solution prepared by Pt(NH₃)₄(NO₃)₂ and 1.45 gram of 2.46 wt % K solution prepared by KNO₃ were mixed together. The solution was loaded in a small rotary evaporator. 9 grams of alumina in 40-60 mesh size was added into the solution. The rotary evaporator rotated for 30 minutes at room temperature, followed by drying with jacketed ambient-pressure steam. The dried material was dried at 100° C. overnight before further calcination in air at 524° C. for 2 hrs. The catalyst is designated Catalyst A with 0.04 wt % Pt and 0.4 wt % K.

### Example 2

A catalyst with 0.06 wt % Pt and 0.4 wt % K on alumina was prepared according to Example 1 preparation procedures and conditions except Pt and K loading were adjusted to obtain 0.06 wt % Pt and 0.4 wt % K on alumina. The catalyst is designated Catalyst B

### Example 3

A catalyst with 0.02 wt % Pt and 0.3 wt % K on alumina was prepared according to Example 1 preparation procedures and conditions except Pt and K loading were adjusted to obtain 0.02 wt % Pt and 0.3 wt % K on alumina. The catalyst is designated Catalyst C.

### Example 4

47.36g of H₂O, 5.94g of 0.15 wt % Pt solution prepared by Pt(NH₃)₄(NO₃)₂, and 3.62g of 2.46 wt % K solution prepared by KNO₃ were mixed together. The solution was loaded in a small rotary evaporator. 30 grams of alumina extrudates was added into the solution. 0.435g of 5 wt % NH₄OH was added to adjust solution pH to 9. Then the rotary evaporator rotated for 30 minutes at room temperature, followed by drying with jacketed ambient-pressure steam. The dried material was dried at 100° C. overnight before further calcination in air at 524° C. for 2 hrs. The calcined catalyst was reduced in pure H₂ at 620° C. for 2 hrs. The prepared catalyst was sized to 40-60 mesh for testing. The catalyst is designated as Catalyst D with 0.03 wt % Pt and 0.3 wt % K.

### Example 5

1.40g of H₂O, 2.43g of 0.145 wt % Pt solution prepared by Pt(NH₃)₄(NO₃)₂ and 0.62 gram of 3 wt % Na solution prepared by NaNO₃ were mixed together. The mixed solution was added to 11.7g of spray-dried alumina support by incipient wetness impregnation technique. The Pt and Na-impregnated support was loaded in a ceramic dish and placed in oven at 100° C. for 6 hrs before further calcination in air at 524° C. for 2 hrs. The catalyst is designated as Catalyst E with 0.03 wt % Pt and 0.17 wt % Na.

### Example 6

A catalyst with 0.03 wt % Pt and 0.3 wt % K on alumina was prepared according to Example 5 preparation procedures and conditions except KNO₃ was used instead of NaNO₃ and Pt and K loading was adjusted to obtain 0.03 wt % Pt and 0.3 wt % K on alumina. The catalyst is designated as Catalyst F.

### Example 7

Catalyst performance evaluation system: catalyst evaluation was carried out in a fixed-bed reactor system at 2.7 hr⁻¹ weight-hourly space velocity (WHSV), 620° C., and ambient pressure with a feed containing H₂/propane mole ratio of 0.17. 0.4g of a catalyst was loaded in a quartz reactor with 3.85 mm ID. Before the propane dehydrogenation reaction, the catalyst was heated in a nitrogen atmosphere and then treated in a gas mixture with a composition of 25 mol % steam, 2.5 mol % O₂, 3.9 mol % CO₂, and balance N₂ at a set temperature in the range between 690-750° C. for 5-13 min. After the treatment, the reactor was purged with dry N₂ and cooled down to 620° C. before the feed containing H₂ and propane was switched into the reactor. The reaction products were analyzed by transmission IR-detector and GC for 5-13 min. After the propane dehydrogenation reaction, the reactor was purged with dry N₂ and was ready for the next cycle of catalyst treatment/regeneration and propane dehydrogenation reaction.

Propane dehydrogenation in specified examples took place in the presence of small amount of moisture. H₂ and propane during the propane dehydrogenation step went through a water saturator to carry a specified level of moisture into the reactor.

Table 1 compares the performance of propane dehydrogenation evaluated by the catalyst performance evaluation system over Catalysts A, B, C, and D with different Pt loading and K loading. Table 1 includes the propane conversion (%) and propylene selectivity (mol %) at 0.65 min time-on-stream calculated from the product distribution analyzed by transmission IR detector. The catalysts were compared after they were regenerated at 750° C. for 13 min before carrying out the propane dehydrogenation at 620° C. No H₂ reduction was carried out after regeneration and before propane dehydrogenation.

Contrary to the prior-art knowledge, it is unexpected that a catalyst with higher Pt loading (e.g. 0.06% Pt) has lower activity than a catalyst with lower Pt loading (e.g. 0.2% Pt).

TABLE 1
(Performance comparison of Catalyst A, B, C, and D)

| | | Propane | Propylene |
|---|---|---|---|
| | | conversion (%) | selectivity (mol %) |
| | Target Pt and K | at 0.65 min | at 0.65 min |
| Catalysts | loading | time-on-stream | time on stream |
| Catalyst A | 0.04% Pt, 0.4% K | 46.9 | 93.9 |
| Catalyst B | 0.06% Pt, 0.4% K | 36.0 | 94.4 |
| Catalyst C | 0.02% Pt, 0.3% K | 41.6 | 93.9 |
| Catalyst D | 0.03% Pt, 0.3% K | 48.8 | 93.2 |

Table 2 compares the performance of catalysts with different alkali elements. The performance comparison was compared after regeneration at 750° C. for 5 min. Catalyst E with 0.03% Pt and 0.17 wt % Na has slightly lower propylene selectivity and activity than Catalyst F with 0.03% Pt and 0.3% K.

TABLE 2
(Performance comparison of Catalyst E and F)

| | | Propane | Propylene |
|---|---|---|---|
| | | conversion (%) | selectivity (mol %) |
| | Target Pt and K | at 0.65 min | at 0.65 min |
| Catalysts | loading | time-on-stream | time on stream |
| Catalyst E | 0.03% Pt, 0.17% Na | 30.7 | 89.8 |
| Catalyst F | 0.03% Pt, 0.3% K | 37.9 | 93.1 |

Catalyst aging system: To evaluate the catalyst performance after many cycles of regeneration/propane dehydrogenation reaction, a reactor system was used for aging catalysts. The system used quartz reactors with 8 mm ID. The catalysts went through cycles of regeneration and propane dehydrogenation reaction. Regeneration was at 690-750° C. in 25% steam-3.2% 02-5.2% CO₂-balance N₂ for 3 min. The propane dehydrogenation was carried out with pure propane for 3.5 min at 620° C. at ambient pressure. Between regeneration and reaction steps, dry N₂ purge was applied. The temperature ramp rate from reaction temperatures to regeneration temperatures was 5° C. /min, while the ramp rate from the regeneration temperature to the reaction temperature is 10° C. /min. The catalysts were unloaded after a desired amount of cycles were completed. Their performance was evaluated at the catalyst performance evaluation system.

### Example 8

According to Example 6 preparation procedures and conditions, three catalysts with 0.03% Pt and 0.3% K loading were prepared on three aluminas with different surface areas. These catalysts are designated as Catalyst G, H, and I. These catalysts were aged in the catalyst aging system for 220, 104, and 134 cycles before being evaluated in the testing system.

Table 3 compares the performance of the aged Catalyst G, H, and I after regeneration at 750° C. for 5 min before propane dehydrogenation. The catalyst with higher surface area has higher propane conversion.

TABLE 3
(Performance comparison of aged Catalyst G, H, and I)

| | | Initial alumina | Propane | Propylene |
|---|---|---|---|---|
| | Aging | surface area | conversion | selectivity |
| Catalyst | cycles | (m2/g) | (%) | (mol %) |
| Catalyst G | 220 | 145 | 48.4 | 92.0 |
| Catalyst H | 104 | 114 | 43.9 | 94.3 |
| Catalyst I | 134 | 113 | 31.3 | 92.1 |

### Example 9

57g of alumina was impregnated with the mixed solution of 0.0417g of SnCl₂ solution with 52.6 wt % Sn and DI H₂O, followed by calcination in air at 650° C. in air for 4 hours to prepare a support with 0.035 wt % Sn. 25g of Sn-loaded support was further impregnated in a small rotary evaporator with a Pt and K solution prepared with chloroplatinic acid (CPA) solution and KOH solution. The rotary evaporator rotated for 1 hr at room temperature, followed by drying with jacketed ambient-pressure steam or heated glycol liquid. The dried material was further dried at 100° C. overnight before calcination in air mixed with HCl/H₂O and Cl₂/N₂ gases at 524° C. for 4 hrs. The obtained material was reduced in pure H₂ at 620° C. for 2 hrs. The prepared catalyst was sized to 40-60 mesh for testing. The catalyst is designated as Catalyst J with 0.03 wt % Pt, 0.035% Sn, and 0.3% K. The catalyst was tested with multiple cycles of regeneration and propane reaction. As shown in Table 4, shorter propane regeneration times are clearly not sufficient to fully recover activity and selectivity over Catalyst J.

TABLE 4
(Catalyst J performance at different regeneration conditions)

| | Propane conversion | Propylene selectivity |
|---|---|---|
| Regeneration | (%) at 0.65 minute | (mol %) at 0.65 minute |
| conditions | time-on-stream | time-on-stream |
| 750 C., | 48.6 | 93.4 |
| 13 min | | |
| 750 C., | 38.2 | 91.1 |
| 5 min | | |

### Example 10

15g of silica-alumina (Siralox 1.5 from Sasol, containing 1.5% SiO₂) was impregnated with Pt and K solution prepared from Pt(NH₃)₄(NO₃)₂ and KNO₃ according to Example 5 impregnation procedures. The Pt-K loaded material was dried and calcined at 750° C. for 2 hrs. The catalyst is designated Catalyst K with 0.03% Pt and 0.3% K on Siralox 1.5. Catalyst K was also subjected to aging in the aging system for 468 cycles. Table 5 compares Catalyst K performance when it was fresh or it went through 468 cycles. The catalyst performance was evaluated after fresh or aged Catalyst K was regenerated at 750° C. for 5 min before propane dehydrogenation reaction. It is clear that Catalyst K has much lower activity and loses some propylene selectivity in successive regeneration cycles.

TABLE 5
(Catalyst K performance after various regeneration/propane
dehydrogenation cycles)

| | Propane conversion | Propylene selectivity |
|---|---|---|
| Aging | (%) at 0.65 minute | (mol %) at 0.65 minute |
| cycles | time-on-stream | time-on-stream |
| 0 | 50.6 | 93.8 |
| 468 | 36.7 | 91.4 |

### Example 11

According to Example 6 preparation procedures and conditions, a catalyst supported on a spray-dried alumina containing 1.5 wt % TiO₂ was prepared. The catalyst is designated as Catalyst L with 0.03% Pt and 0.3% K on spray-dried alumina containing 1.5 wt % TiO₂.

The propane dehydrogenation performance of Catalyst L containing 1.5 wt % TiO₂ was compared with Catalyst H without TiO₂. The performance was evaluated after the catalysts were subjected to regeneration at 750° C. for 5 min. As shown in Table 6, Catalyst L containing 1.5% TiO₂ is inferior to Catalyst K containing no TiO₂.

TABLE 6
(Performance comparison of Catalyst H and L)

| | Propane conversion | Propylene selectivity |
|---|---|---|
| | (%) at 0.65 minute | (mol %) at 0.65 minute |
| Catalyst | time-on-stream | time-on-stream |
| Catalyst H | 45.2 | 93.1 |
| Catalyst L | 25.3 | 85.8 |

### Example 12

According to Example 6 preparation procedures and conditions, a catalyst supported on a spray-dried alumina containing 1.2 wt % boron was prepared. The catalyst is designated as Catalyst M with 0.03% Pt and 0.3% K on spray-dried alumina containing 1.2 wt % boron.

Catalyst M was subjected to aging in the aging system for 134 cycles before testing. The performance of Catalyst M was evaluated after Catalyst M was regenerated at 750° C. for 5 min. Compared with Catalyst H, it is clear that boron-containing catalyst has much lower activity and selectivity than the catalyst without boron such as Catalyst H.

TABLE 7
(Performance comparison of Catalyst M and H)

| | | Propane | Propylene |
|---|---|---|---|
| | Aging | conversion | selectivity |
| Catalyst | cycles | (%) | (mol %) |
| Catalyst H | 104 | 43.9 | 94.3 |
| Catalyst M | 134 | 17.2 | 82.7 |

### Example 13

A catalyst without Sn was prepared similarly as Catalyst J (0.03 wt % Pt-0.035 wt % Sn-0.3 wt % K on alumina). The Pt precursor was chloroplatinic acid (CPA) and K precursor was KOH. The Pt and K impregnated material was dried at 100C overnight before further calcination in air mixed with HCl/H₂O and Cl₂/N₂ streams at 524° C. for 4 hrs. The obtained material was reduced in pure H₂ at 620° C. for 2 hrs. The prepared catalyst was further steamed at 700° C. for 6 hours in the presence of air and 25 mol % steam. The prepared catalyst is designated as Catalyst 0 with 0.03% Pt and 0.3% K on an alumina support containing Cl.

Comparing Catalyst D and Catalyst 0, the catalyst prepared with Cl-containing Pt precursor and oxy-chlorination has much lower propylene selectivity than Catalyst D, and surprisingly, also have lower propane conversion. Catalyst D and Catalyst 0 were both regenerated at 750° C. for 13 min before propane dehydrogenation reaction.

TABLE 8
(Performance comparison of Catalyst D and Catalyst O)

| | Propane conversion | Propylene selectivity |
|---|---|---|
| | (%) at 0.76 min | (mol %) at 0.76 min |
| Catalyst | time-on-stream | time-on-stream |
| Catalyst D | 49.3 | 92.8 |
| Catalyst O | 38.6 | 91.2 |

### Example 14

125cc of alumina extrudate with surface area of 125 m²/g was impregnated with the mixture of 19.4g of 10 wt % LiNO₃ solution, 19.4g of 10 wt % HNO₃ solution, and 221g of DI water. After the impregnation, the dried Li-alumina was calcined in air at 850° C. to prepare a support with 1.5 wt % Li. 20g of calcined Li-alumina support was further impregnated with a Pt solution prepared by mixing 0.18g of 3.3% CPA (H₂PtCl₆) solution, 1.8g of 36.5 wt % HCl solution, and 35.6 g of DI water. The support and Pt solution were mixed in a small rotary evaporator. The rotary evaporator rotated for 1 hr at room temperature, followed by drying with jacketed ambient-pressure steam. The dried material was dried at 100° C. overnight before further calcination in air at 524° C. for 2 hrs. The calcined catalyst was reduced in pure H₂ at 620° C. for 2 hrs. The prepared catalyst was sized to 40-60 mesh for testing. The catalyst is designated as Catalyst P with 0.03 wt % Pt and 1.5 wt % Li. Propane dehydrogenation was evaluated after regeneration at 750° C. for 13 minutes. At 0.55 minutes on stream propane conversion was 52.5% and propylene selectivity was 92.7%. At 1.5 minutes on stream conversion was 35.0% with propylene selectivity of 93.6%. As comparison, Catalyst F, tested at the same conditions had propane conversion of 50.8% and propylene selectivity of 92.7% at 0.55 min on stream; and 43.5% propylene conversion and selectivity of 93.2% at 1.5 minutes on stream.

### Example 15

According to Example 6 preparation procedures and conditions, three catalysts with 0.03% Pt and 0.3% Ca, or 0.03% Pt and 0.66 wt % Sr, or 0.03% Pt and 1.22 wt % Ba, respectively, were prepared. They are designated as Catalyst Q, R, and S. Before testing, they were subjected to aging in the aging system for 268, 134, and 134 cycles respectively. They were tested in propane dehydrogenation with the presence of 4000-5000 mole ppm moisture after regeneration at 750° C. for 5 minutes. Performance at 0.56 min on stream is shown in table 9.

TABLE 9
(Performance comparison of Catalyst Q, R, and S)

| | | Propane | Propylene |
|---|---|---|---|
| | Aging | conversion | selectivity |
| Catalyst | cycles | (%) | (mol %) |
| Catalyst Q | 268 | 47.4 | 92.7 |
| Catalyst R | 134 | 49.0 | 91.5 |
| Catalyst S | 134 | 43.4 | 93.0 |

### Example 16

A catalyst was prepared similarly to catalyst Q, but on a spray dried alumina support that contained 0.3 wt % Ca and had BET surface area of 126 m²/g. Additional Ca and Pt was added by incipient wetness impregnation for total of 0.4 wt % Ca and 0.03 wt % Pt. This catalyst is designated catalyst T. Before testing, the catalyst was subjected to aging in the aging system for 134 cycles. It was tested in propane dehydrogenation with the presence of 4000-5000 mole ppm moisture. After regeneration at 750° C. for 5 minutes propane conversion was 47.13% at 0.65 minutes on stream and propylene selectivity was 92.52%.

### Example 17

A catalyst was prepared similarly to catalyst Q, but on a spray dried alumina support that had lower surface area of 88 m²/g, and higher theta index. The catalyst is designated as Catalyst U. Before testing, the catalyst was subjected to aging in the aging system for 134 cycles. It was tested in propane dehydrogenation with the presence of 4000-5000 mol ppm moisture. Owing to the low surface area of the support, the catalyst was not as active. After regeneration at 750° C. for 5 minutes propane conversion was 40.29% and propylene selectivity was 93.38% evaluated at 0.65 minutes on stream. In a subsequent reaction cycle after regeneration at 700° C. for 5 minutes, propane conversion was 32.8% after 0.55 minutes on stream and selectivity was 92.71%.

Table 10 shows the total integrated alumina peaks, the “theta-index” and “alpha-index” for the alumina supports for described example catalysts, along with the NIST 676A standard and a sample that was primarily theta alumina.

TABLE 10
Theta-Index or Alpha-Index of example catalysts

| | AI Integrated peaks | Theta-Index | |
|---|---|---|---|
| | Corrected for X-ray | (Specified Alpha | |
| Samples | tube | peak = 1.00) | Alpha-Index |
| NIST 676A alpha | 25.707 | 1.000 | 1.000 |
| alumina standard | | | |
| Theta Alu mina | 3.900 | 0.152 | 0.02 |
| Catalyst U | 1.136 | 0.044 | — |
| Catalyst T | 0.478 | 0.019 | — |
| Catalyst I | 0.533 | 0.021 | — |
| Catalyst S | 0.232 | 0.009 | — |
| Catalyst G | 0.181 | 0.000 | — |

### Example 18

A catalyst was prepared in the same manner as Catalyst D, but using Ca instead of K, with 2.3 wt % Ca from calcium nitrate. The catalyst was tested in the catalyst testing apparatus. Catalyst is designated catalyst V. Performance of catalyst V after regeneration at 750° C. for 5 min before propane dehydrogenation was evaluated. After 0.65 minutes on stream propane conversion was 38.08% and propylene selectivity was 94.97%. After regeneration at 750° C. for 30 minutes followed by reduction in hydrogen at 620° C. for 10 minutes, followed by testing propane dehydrogenation at 620° C., conversion at 0.65 min on stream was 25.2% and selectivity to propylene was 90.40%. Performance after a reduction step is clearly worse than without an intervening reduction step.

### Example 19

A catalyst was prepared similar to catalyst F but using a spray dried alumina support containing 2% phosphorous and no potassium was added. The catalyst also contained 0.03% Pt. Catalyst is designated catalyst W. Before testing, the catalyst was subjected to aging in the aging system for 134 cycles. The catalyst was tested in the catalyst testing apparatus. Performance of catalyst W after regeneration at 750C for 5 min was evaluated. After 0.65 minutes on stream propane conversion was 33.8% and propylene selectivity was 90.8%.

### Example 20

A catalyst was prepared similar to catalyst F but using a spray dried alumina support containing 1% magnesium. Potassium and Pt were added by impregnation such that the final catalyst contained 0.03% Pt, 0.3% K and 1% Mg. Catalyst is designated catalyst X. Before testing, the catalyst was subjected to aging in the aging system for 134 cycles. The catalyst was tested in the catalyst testing apparatus. Performance of catalyst X after regeneration at 750C for 5 min was evaluated. After 0.76 minutes on stream propane conversion was 41.7% and propylene selectivity was 93.1%.

### Example 21

The alumina catalyst supports of some of the above catalysts were subjected to a steam aging treatment in 25 mol % steam at 780 C for 23 hours. Some of these alumina supports contained additives. In addition, a spray dried alumina support containing 0.47% Si was also subjected to the same test. The BET surface area before and after treatment was determined for each alumina catalyst support. The change in surface area for each support is reported in table 11.

TABLE 11

| | | Amount (wt % | % surface |
|---|---|---|---|
| Additive | catalyst # | of element) | area loss |
| None | Q, R, S | None | 18.8% |
| B | M | 1.20% | 14.7% |
| Ca | T | 0.30% | 8.73% |
| Mg | X | 1% | 10.8% |
| P | W | 2% | 3.36%, 4.70%* |
| Si | — | 0.47% | 4.17% |

*repeat steam aging experiments

### SPECIFIC EMBODIMENTS

While the following is described in conjunction with specific embodiments, it will be understood that this description is intended to illustrate and not limit the scope of the preceding description and the appended claims.

A first embodiment of the invention is a process for dehydrogenating a paraffinic hydrocarbon comprising sending the paraffinic hydrocarbon to a fluidized bed reactor to be contacted at dehydrogenation reaction conditions with a catalyst composition comprising less than about 0.0999 wt % platinum and about 0.05-2.5 wt % Group I or Group II elements or a mixture thereof wherein the catalytic composition is prepared without addition of tin, gallium, indium, germanium or lead. An embodiment of the invention is one, any or all of prior embodiments in this paragraph up through the first embodiment in this paragraph wherein the catalytic composition comprises less than about 100 ppm by weight of tin, gallium, indium, germanium, lead and chromium. An embodiment of the invention is one, any or all of prior embodiments in this paragraph up through the first embodiment in this paragraph wherein the platinum and the Group I and Group II elements are present at an atomic ratio of about 1:20 to 1:200. An embodiment of the invention is one, any or all of prior embodiments in this paragraph up through the first embodiment in this paragraph wherein during operation of the process the catalytic composition comprises less than about 1000 ppm by weight chloride. The process in claim 1 wherein the the Group I or Group II elements comprise potassium or calcium. The process in claim 1 wherein the support for the catalytic composition comprises alumina. The process in claim 7 wherein the support comprises gamma alumina and has theta index of less than 0.04. An embodiment of the invention is one, any or all of prior embodiments in this paragraph up through the first embodiment in this paragraph wherein the catalytic composition is in a form of particles comprising a particle size of 20-200 micrometers. An embodiment of the invention is one, any or all of prior embodiments in this paragraph up through the first embodiment in this paragraph wherein the catalytic composition comprises particles with a median particle size of 50-150 micrometers. An embodiment of the invention is one, any or all of prior embodiments in this paragraph up through the first embodiment in this paragraph wherein the catalyst composition comprises particles having a surface area of about 85 to about 140 m²/g. An embodiment of the invention is one, any or all of prior embodiments in this paragraph up through the first embodiment in this paragraph wherein the catalyst composition has a bulk density of about 0.7-1.1 g/cm³. An embodiment of the invention is one, any or all of prior embodiments in this paragraph up through the first embodiment in this paragraph wherein the catalyst composition comprises more than 0.0050% by weight platinum and less than 0.0600% by weight platinum. An embodiment of the invention is one, any or all of prior embodiments in this paragraph up through the first embodiment in this paragraph wherein the catalyst composition comprises less than 0.04 micromole of Pt per m² of surface area. An embodiment of the invention is one, any or all of prior embodiments in this paragraph up through the first embodiment in this paragraph wherein the catalyst composition comprises from about 25 to 130 micromoles of the Group I or Group II elements per gram of catalyst composition. The dehydrogenation process of claim 1 wherein the catalyst is contacted with a stream containing a paraffin at dehydrogenation conditions and then passed to a regeneration zone wherein the catalyst is regenerated at regeneration conditions, wherein the regeneration conditions consist of contacting the catalyst with a stream comprising oxygen. An embodiment of the invention is one, any or all of prior embodiments in this paragraph up through the first embodiment in this paragraph wherein the regenerator comprises a regenerator burn zone containing 0.5-20 mole % oxygen, 10-30 mole % steam and 2-8 mole % carbon dioxide. An embodiment of the invention is one, any or all of prior embodiments in this paragraph up through the first embodiment in this paragraph comprising first regenerating the catalyst composition to produce a regenerated catalyst composition and then sending the regenerated catalyst composition to a fluidized bed dehydrogenation reactor directly without first undergoing a reduction reaction. An embodiment of the invention is one, any or all of prior embodiments in this paragraph up through the first embodiment in this paragraph wherein the regenerated catalyst composition is first contacted with nitrogen or an inert gas and then sent to the fluidized bed dehydrogenation reactor. An embodiment of the invention is one, any or all of prior embodiments in this paragraph up through the first embodiment in this paragraph wherein the regenerated catalyst composition is sent to the fluidized bed dehydrogenation reactor without contact with a halogen to disperse platinum. An embodiment of the invention is one, any or all of prior embodiments in this paragraph up through the first embodiment in this paragraph wherein catalyst is regenerated and has a temperature of 600 to 800° C. before returning to the reactor. An embodiment of the invention is one, any or all of prior embodiments in this paragraph up through the first embodiment in this paragraph wherein the fluidized bed reactor produces propylene and hydrogen at a bulk average temperature of about 550 to 680° C. The process in claim 1 wherein the average catalyst residence time in the fluidized bed reactor is between 30 seconds and 5 minutes.

A second embodiment of the invention is a process for dehydrogenating a paraffinic hydrocarbon comprising sending said paraffinic hydrocarbon to a fluidized bed reactor to be contacted at dehydrogenation reaction conditions with a catalyst composition comprising less than about 0.0999 wt % platinum and about 0.05-2.5 wt % calcium.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for dehydrogenating a paraffinic hydrocarbon comprising sending said paraffinic hydrocarbon to a fluidized bed reactor to be contacted at dehydrogenation reaction conditions with a catalyst composition comprising less than about 0.0999 wt % platinum and about 0.05-2.5 wt % Group I or Group II elements or a mixture thereof wherein said catalyst composition is prepared without addition of tin, gallium, indium, germanium, chromium, or lead.

2. The process of claim 1 wherein said catalyst composition comprises less than about 100 ppm by weight of tin, gallium, indium, germanium, lead, and chromium.

3. The process of claim 1 wherein said platinum and said Group I and Group II elements are present at an atomic ratio of about 1:20 to 1:200.

4. The process of claim 1 wherein during operation of said process said catalytic composition comprises less than about 1000 ppm by weight chloride.

5. The process in claim 1 wherein the said Group I or Group II elements comprise potassium or calcium.

6. The process in claim 1 wherein the support for said catalyst composition comprises alumina.

7. The process in claim 6 wherein the support comprises gamma alumina and has theta index of less than 0.04.

8. The process of claim 1 wherein said catalytic catalyst composition comprises particles with a median particle size of 50-150 micrometers.

9. The process of claim 1 wherein said catalyst composition comprises particles having a surface area of about 85 to about 140 m2/g.

10. The process of claim 1 wherein said catalyst composition comprises more than 0.0050% by weight platinum and less than 0.0600% by weight platinum.

11. The process of claim 1 wherein said catalyst composition comprises less than 0.04 micromole of Pt per m2 of surface area.

12. The process of claim 1 wherein said catalyst composition comprises from about 25 to 130 micromoles of said Group I or Group II elements per gram of catalyst composition.

13. The dehydrogenation process of claim 1 wherein the catalyst is contacted with a stream containing a paraffin at dehydrogenation conditions and then passed to a regeneration zone wherein the catalyst is regenerated at regeneration conditions, wherein the regeneration conditions consist of contacting the catalyst with a stream comprising oxygen.

14. The process of claim 13 wherein said regenerator comprises a regenerator burn zone containing 0.5-20 mole % oxygen, 10-30 mole % steam and 2-8 mole % carbon dioxide.

15. The process of claim 13 comprising first regenerating said catalyst composition to produce a regenerated catalyst composition and then sending said regenerated catalyst composition to a fluidized bed dehydrogenation reactor directly without first undergoing a reduction reaction.

16. The process of claim 13 wherein said regenerated catalyst composition is first contacted with nitrogen or an inert gas and then sent to said fluidized bed dehydrogenation reactor.

17. The process of claim 13 wherein catalyst is regenerated and has a temperature of 600 to 800° C. before returning to the reactor.

18. The process of claim 1 wherein the paraffinic hydrocarbon is propane, and the fluidized bed reactor produces propylene and hydrogen at a bulk average temperature of about 550 to 680° C.

19. The process in claim 1 wherein the average catalyst residence time in the fluidized bed reactor is between 30 seconds and 5 minutes.

20. A process for dehydrogenating a paraffinic hydrocarbon comprising sending said paraffinic hydrocarbon to a fluidized bed reactor to be contacted at dehydrogenation reaction conditions with a catalyst composition comprising less than about 0.0999 wt % platinum and about 0.05-2.5 wt % calcium.
